(19) United States
(12) Patent Application Publication
(10) Pub. No.: US 2004/0050737 A1
(43) Pub. Date: Mar. 18, 2004

(54) Hygienic wipes

(76) Inventors: Donna Burton, Chesterfield (GB); Kerry Clarke, Chesterfield (GB); Kathy Clark, Chesterfield (GB)

Correspondence Address: WILLIAM COLLARD, COLLARD & ROE, P.C., 1077 NORTHERN BOULEVARD, ROSLYN, NY, 11576, US

(21) Appl. No.: 10/333,876

(22) PCT Filed: Jul. 27, 2001
(86) PCT No.: PCT/GB01/03346

(30) Foreign Application Priority Data
Jul. 27, 2000 (GB) 0018393.9

(51) Int. Cl. D04H 1/00; A61B 19/02

(52) U.S. Cl. 206/438; 442/327

(57) ABSTRACT

A hygienic wipe comprises a moisture retaining pad (10), of for example multi ply biodegradable gauze tissue material impregnated with distilled water. Pads (10) may be encapsulated in an array of sachets (11) formed from acoustically or thermally weldable material (15, 19) welded along lines (13) to form air and water tight bubbles to contain the pads until required or individually wrapped.

This invention relates to the improvements in hygienic wipes, that is pads or the like used for cleaning surfaces or skin in clinical and every day domestic uses.

It is known to provide wipes on the form of pads of fabric or multi-layered paper tissue. It is also known to impregnate such pads with biocidal, disinfectant or astringent liquid, alcohol, or potions.

For many uses, however, such as in connection with young children, it is not desirable to bring liquids of the types mentioned into contact with either the children themselves or with articles with which the children may be expected to come into contact, particularly toys, teething rings, dummies and other comforts. Many of these impregnating materials are either unpleasant to taste, or are actually harmful if ingested in a sufficient quantity.

It is an object of the invention to provide a hygienic wipe which provides a ‘wet’ wipe for cleaning purposes, but is not impregnated with a material which may be harmful or unpleasant if ingested in excess, or if allowed to contact skin or internal membranes, as may be the case with the example chlorinated disinfectants.

In accordance with the invention, a hygienic wipe comprises a pad; for example a multi layer structure of gauzes or tissue, which is impregnated with sterilised water. In the case of ‘wet’ wipes, it is usual to use a non-drying drying impregnating material, which remains moist for some time following exposure.

In accordance with a second feature of the invention, each pad is preferably sealed in an air and water-proof envelope or sachet following impregnation, to ensure that the pad remains moist until required for use, at which time the envelope or sachet will be opened and the pad removed and used.

The envelopes or sachets may be individual wrappings, or be provided in sheets consisting of several such envelopes connected together along severance lines.

The envelopes or sachets are preferably of a non-water or air permeable material, to ensure that the pads encapsulated within them remain at the required degree of moistness until opened for use. The sachet material is also preferably weldable by heat or acoustic methods during the encapsulation process.

The degree of moistness of the pads may be selected e.g. to ensure that moisture does not leak from the pad unless pressure is applied to the pad, but sufficient to ensure that moisture does seep from the pad when he pad is pressed against a surface, to ensure that a moist wipe is provided.

An embodiment of the invention will now be described by way of example with reference to the accompanying drawings, wherein:—

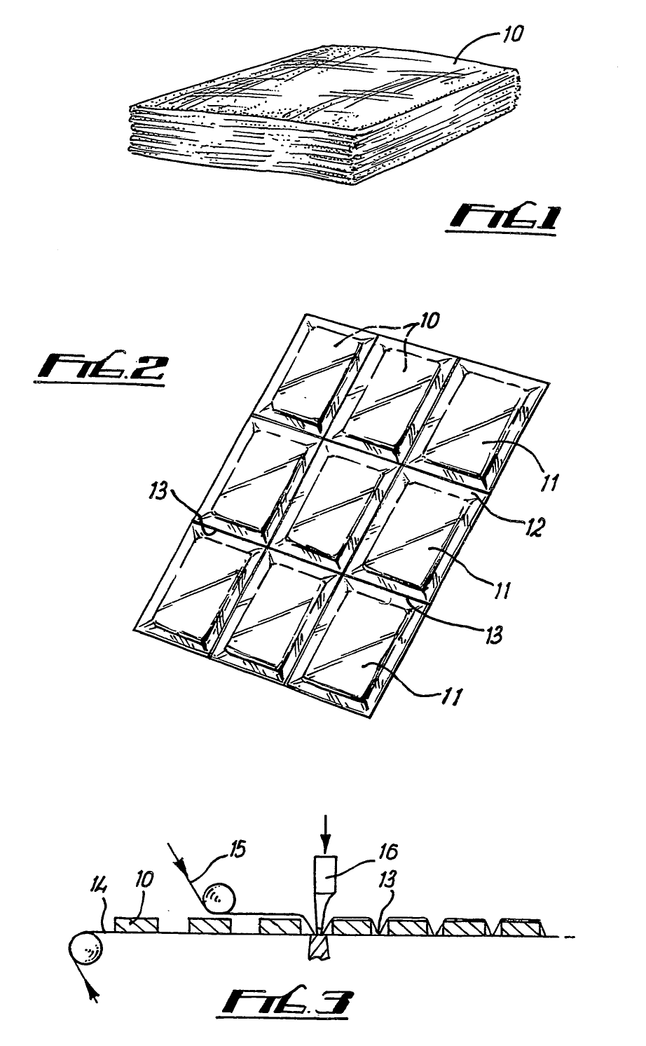

FIG. 1 is a perspective view of a hygienic wipe pad according to the invention;

FIG. 2 shows an array of pads according to FIG. 1, packaged in individual sachets forming a sheet of such sachets; and

FIG. 3 is a diagram illustrating a method of packaging the pads to form a sheet of sachets.

As shown in FIG. 1, a hygienic wipe comprises a pad 10, consisting of multiple layers of superposed tissues of biodegradable gauze. This pad 10 is impregnated with sterilised water, to achieve a degree of moistness whereby the water does not leak from the pad, but is expressed by exerting pressure on the pad, to moisten any surface to which the pad is applied.

As shown in FIG. 2, a plurality of pads 10 may be encapsulated in sachets or envelopes 11 of waterproof non-air permeable material, which may be formed into sheets 12 of bliter packaging or the like, separated by perforated or scored lines of severance 13, so that the sachets 11 can be removed as required and opened for use.

In FIG. 3, pads 10 are placed on a lower sheet 14 of the weldable waterproof non-air permeable material, and a second sheet is of the same material is placed over the pads 10. A welding knife or die 16 which may for example be a thermal or an acoustic welding head joins the sheets around the pads to form the sachets 11. The severance lines 13 may be formed at the same time, or in a subsequent step using a scoring or perforating tool (not shown).

Alternatively the pads may be individually wrapped.

The air and watertight packaging ensures that the pads 10 remain moist, at the correct degree of moistness until required for use, as moisture is neither added to or removed from the pads.

Sterilised water has a number of important advantages over pads impregnated with other materials such a lotions, moisturisers, disinfectants, alcohol, biocides or astringents, since it enable a clean wipe to be made but does not itself pose any hazard-either as an allergen or low-level poison, nor does it have any unpleasant taste. This is a great advantage in clinical and domestic environments, particularly where young children are being attended to. For example, a toy or comforter which has been dropped can be wiped with a sterile water wipe without anxiety that the impregnating material may cause a child harm when the toy etc is later put by the child in his her mouth. The wipe can also be used for wiping sensitive areas, such as the eyes with out risk or discomfort, or for cleaning nipples prior to breast feeding, to remove lint from pads, or dried milk.

1. A hygienic wipe comprising a porous pad adapted to retain a liquid, and release the liquid when subjected to pressure, the porous pad being impregnated with sterilised water.

2. A wipe according to claim 1, wherein said porous pad comprises a multi-layer structure of biodegradable gauze tissues.

3. A wipe according to claim 1 or 2, wherein said pad is sealed in an air and water tight envelope or sachet following impregnation of the pad.

4. A plurality of wipes each according to any one of claims 1 to 3, enclosed in an array of sachets of air and water-tight material disposed in sheets consisting of several such sachets connected together along severance lines.

5. A plurality of wipes according to claim 4 wherein the sachet material is weldable by heat or acoustic welding during encapsulation.

6. A wipe according to claim 3, wherein each pad is individually wrapped.

7. A hygienic wipe substantially as hereinbefore described, with reference to and as illustrated in the accompanying drawings.
